(19) United States
(12) Patent Application Publication
(10) Pub. No.: US 2023/0154569 A1
(43) Pub. Date: May 18, 2023

(54) METHODS OF FUNCTIONALITY SCREENING BIOLOGICAL SEQUENCE FRAGMENTS

(71) Applicant: Massachusetts Institute of Technology, Cambridge, MA (US)

(72) Inventors: Kevin Michael Esvelt, Cambridge, MA (US); Dana Gretton, Cambridge, MA (US)

(73) Assignee: Massachusetts Institute of Technology, Cambridge, MA (US)

(21) Appl. No.: 17/793,968

(22) PCT Filed: Jan. 23, 2021
(86) PCT No.: PCT/US2021/014814

§ 371 Date: Jul. 20, 2022

Related U.S. Application Data
(60) Provisional application No. 62/965,138, filed on Jan. 23, 2020.

(51) Int. Cl. G16B 35/20; G16B 50/00

(52) U.S. Cl. CPC G16B 35/20; G16B 50/00

(57) ABSTRACT

The invention relates, in part, to methods of accurately and reliably detecting biological sequences corresponding to a particular function.

### RELATED APPLICATIONS

This application claims the benefit under 35 U.S.C. § 119(e) of U.S. Provisional application Ser. No. 62/965,138 filed Jan. 23, 2020, the disclosure of which is incorporated by reference herein in its entirety.

### FIELD OF THE INVENTION

The invention relates, in part, to methods for detecting biological sequences corresponding to a particular biological function while minimizing the incorrect detection of sequences with unrelated functions.

### BACKGROUND OF THE INVENTION

Governments strongly recommend that industry screen nucleic acid synthesis orders to prevent the construction of biological weapons [Diggans, J. and E. Leproust. Frontiers in Bioengineering and Biotechnology 7 (April): 86 (2019)]. Many companies voluntarily screen against agents identified by nations and international groups [see International Gene Synthesis Consortium. (2017) “Harmonized Screening Protocol V2 //genesynthesisconsortium.org/wp-content/uploads/IGSCHarmonizedProtocol11-21-17.pdf.] Current screening methods rely on similarity search algorithms [Altschul et al. Journal of Molecular Biology 215 (3): 403-10 (1990)] to identify sequences similar to those from known bioweapons. These algorithms cannot screen small pieces of nucleic acids that could be assembled into larger pieces. Many innocent sequences are similar enough to be identified by similar search as hazardous, generating false positives that require expert human curation and precluding automated screening. Screening methods that are automated and can be applied to benchtop nucleic acid synthesizers and assemblers, which necessarily cannot rely on human experts to curate false positives, are not available.

### SUMMARY OF THE INVENTION

According to an aspect of the invention, a method of assessing a biological sequence capable of a preselected function is provided, the method including: (a) preselecting a biological molecule, wherein the biological molecule is capable of a function of interest; (b) preparing a testing sequence database comprising a plurality of sequence fragments of the preselected biological molecule, wherein the preselected sequence fragments are a predetermined length; (c) fragmenting the sequence of one or more test biological molecules into lengths equivalent to the predetermined length of the sequence fragments of the preselected biological molecule in the testing sequence database; (d) detecting a presence or absence of a sequence match between the sequence of at least one fragment of the fragmented test biological molecules and at least one of the plurality of sequence fragments of the preselected biological sequence, and (e) acting in response to the detection in (d), wherein the detecting in (d) provides an assessment of the test biological molecule. In some embodiments, the acting in response to (d) includes one of more of: preventing synthesis of the test biological molecule, permitting synthesis of the test biological molecule, sequencing one or more polynucleotide molecules, DNA sequencing, DNA molecule design, polypeptide sequence determination, and further sequence identification steps. In certain embodiments, the method also includes identifying in the testing sequence database one or more sequence fragments of the preselected biological sequence that match one or more sequence fragments, respectively, of a second biological molecule having a biological function unrelated to the biological function of interest of the preselected biological molecule, and removing the identified sequence fragments(s) from the testing sequence database. In certain embodiments, if the presence of a sequence match is detected in (d) the action includes preventing synthesis of the test biological molecule. In some embodiments, a means of preparing the testing sequence database includes: (a) screening the plurality of sequence fragments of the preselected biological sequence molecule against at least one control sequence database, wherein the control sequence database includes a plurality of control sequence fragments of at least one molecule capable of a function of interest unrelated to the function of interest of the preselected biological molecule; (b) identifying the presence of a match between a sequence fragment in the plurality of sequence fragments of the preselected biological molecule and a sequence fragment in the control sequence database that is a fragment of the biological molecule identified as capable of a function unrelated to the function of interest of the preselected biological molecule; and (c) removing from the testing sequence database the sequence fragment of the preselected biological sequence identified as matching the sequence fragment of the biological sequence identified as capable of a function of interest unrelated to the function of interest of the molecule capable of the function of interest. In some embodiments, the preselected biological molecule is a polynucleotide. In certain embodiments, the sequence of the biological molecule is a full-length nucleic acid sequence of the polynucleotide or is a portion of the full-length nucleic acid sequence of the polynucleotide. In certain embodiments, the full-length nucleic acid sequence encodes a protein. In some embodiments, the predetermined length of the sequence fragments of the preselected polynucleotide molecule is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50, 55, 60, 65, 70, 75, 80, 85, 90, 95, 100, 110, 120, 130, 140, 150, 160, 170, 180, 190, 200, or more nucleotides. In some embodiments, the preselected biological molecule includes a polypeptide. In some embodiments, the amino acid sequence of the preselected biological molecule is a full-length amino acid sequence of the polypeptide, or is a portion of the full-length amino acid sequence of the polypeptide. In some embodiments, the predetermined length of the sequence fragments of the preselected polypeptide molecule is 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 91, 2, 93, 94, 95, or more amino acids. In certain embodiments, the plurality of sequence fragments of the preselected biological molecule: (1) includes all or a significant portion of possible fragments or at least one essential fragment of the biological molecule that is capable of the function of interest, and (2) does not comprise sequences found in a biological molecule capable of a function unrelated to the function of the preselected biological molecule. In some embodiments, the control sequence database includes a plurality of control sequence fragments of at least one molecule capable of a function of interest unrelated to the function of interest of the preselected biological molecule. In some embodiments, the control sequence database includes a plurality of control sequence fragments of at least one molecule not capable of the function of interest of the preselected biological molecule. In certain embodiments, the predetermined length is the same for all fragments of the preselected biological molecule. In some embodiments, the predetermined length of the sequence fragments of the preselected biological molecule includes more than one length. In some embodiments, the testing sequence database includes one or more sequence fragments randomly or pseudorandomly selected from sequences of molecules known to be capable of a function different from the preselected molecule's function of interest. In certain embodiments, the randomly or pseudorandomly selected sequence fragments are biased towards sequence regions with greater homology to functionally or phylogenetically related sequences. In certain embodiments, the testing sequence database further includes sequences that are functional equivalents of the plurality of sequence fragments of the preselected biological molecule. In some embodiments, a means for identifying the functional equivalents includes a computational means. In some embodiments, a means for identifying the functional equivalents includes an experimental means. In some embodiments, a computational means for selecting the functional equivalents included in the testing sequence database includes using a classifier based on experimental data to evaluate the accuracy of the computational means. In certain embodiments, a means for selecting the functional equivalents included in the testing sequence database includes inclusion of a minimal number of sequences calculated to achieve a predetermined likelihood of successfully preventing a test sequence from escaping detection. In some embodiments, a means for selecting the functional equivalents included in the testing sequence database includes a random selection method or a pseudorandom selection method. In certain embodiments, the identities of all sequence fragments of one or both of the testing sequence database and the test biological molecule are protected. In some embodiments, a means of the protecting includes application of a cryptographic hash function, wherein the cryptographic hash function deterministically maps the sequence data to a bit string of fixed size using a one-way function. In some embodiments, the application of the cryptographic hash function cannot be reversed without a brute-force search of all possible sequence inputs into the testing sequence database. In some embodiments, the application of the cryptographic hash function further includes use of one or more information keys that must be accessed to attempt the brute-force search. In certain embodiments, the application of the cryptographic hash function requires keys from a plurality of independent sources that must cooperate to compute the hash without any one server gaining access to the sequence data. In certain embodiments, the independent sources comprise independent computer servers. In some embodiments, the method also includes dividing the prepared testing sequence database into two or more partial testing sequence databases, and the prepared testing sequence database used for detecting of the presence or absence of a sequence match is one two or more partial testing sequences databases. In some embodiments, if a sequence match is detected between the partial testing sequence database and one or more fragments of the test biological molecules, the method further includes detecting the presence or absence of a sequence using another of the two or more partial testing sequence databases. In some embodiments, the testing sequence database contains a portion of a larger database of sequence fragments such that the fragments included in the testing sequence database can be rotated frequently or upon a match being discovered.

According to another aspect of the invention, a method of identifying a biological sequence capable of a preselected function is provided, the method including: (a) preselecting a biological molecule, wherein the preselected biological molecule is capable of a function of interest; (b) preparing a testing sequence database comprising a plurality of sequence fragments of the preselected biological molecule, wherein the preselected sequence fragments are a predetermined length; (c) fragmenting the sequence of one or more test biological molecules into lengths equivalent to the predetermined length of the sequence fragments the preselected biological molecule in the testing sequence database; and (d) detecting a presence or absence of a sequence match between the sequence of at least one fragment of the fragmented test biological molecules and at least one of the plurality of sequence fragments of the preselected biological sequence; wherein a means of preparing the testing sequence database includes: (i) screening the plurality of sequence fragments of the preselected biological sequence molecule against at least one control sequence database, wherein the control sequence database includes a plurality of control sequence fragments of at least one molecule capable of a function of interest unrelated to the function of interest of the preselected biological molecule; (ii) identifying the presence of a match between a sequence fragment in the plurality of sequence fragments of the preselected biological molecule and a sequence fragment in the control sequence database that is a fragment of the biological molecule identified as capable of a function unrelated to the function of interest of the preselected biological molecule; and (iii) removing from the testing sequence database the sequence fragment of the preselected biological sequence identified as matching the sequence fragment of the biological sequence identified as capable of a function of interest unrelated to the function of interest of the molecule capable of the function of interest. In some embodiments, the preselected biological molecule is a polynucleotide. In certain embodiments, the sequence of the preselected biological molecule is a full-length nucleic acid sequence of the polynucleotide or is a portion of the full-length nucleic acid sequence of the polynucleotide. In some embodiments, the full-length nucleic acid encodes a protein. In some embodiments, the predetermined length of the sequence fragments of the preselected polynucleotide molecule is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50, 55, 60, 65, 70, 75, 80, 85, 90, 95, 100, 110, 120, 130, 140, 150, 160, 170, 180, 190, 200, or more nucleotides. In certain embodiments, the preselected biological molecule includes a polypeptide molecule. In some embodiments, the sequence of the preselected biological molecule is a full-length amino acid sequence of the polypeptide, or is a portion of the full-length amino acid sequence of the polypeptide. In some embodiments, the predetermined length of the sequence fragments of the preselected polynucleotide molecule is 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 91, 2, 93, 94, 95, or more amino acids. In some embodiments, the plurality of sequence fragments of the preselected biological molecule: (1) includes all or a significant portion of possible fragments of the biological molecule that is capable of the function of interest, and (2) does not comprise sequences not found in a biological molecule capable of a function unrelated to the function of the preselected biological molecule. In certain embodiments, the control sequence database includes a plurality of control sequence fragments of at least one molecule capable of a function of interest unrelated to the function of interest of the preselected biological molecule. In some embodiments, the control sequence database includes a plurality of control sequence fragments of at least one molecule not capable of the function of interest of the preselected biological molecule. In some embodiments, a single length is the predetermined length of the sequence fragments of the preselected biological molecule. In certain embodiments, there is more than one predetermined length of the sequence fragments of the preselected biological molecule. In some embodiments, the testing sequence database includes one or more sequence fragments randomly selected from sequences of molecules known to be capable of a function different from the preselected molecule's function of interest. In some embodiments, the randomly selected sequence fragments are biased towards conserved regions. In certain embodiments, the testing database further includes sequences that are functional equivalents of the plurality of sequence fragments of the preselected biological molecule. In some embodiments, the identities of all sequence fragments of one or both of the testing sequence database and the test biological molecule are protected. In certain embodiments, a means of the protecting includes application of a cryptographic hash function, wherein the cryptographic hash function deterministically maps the sequence data to a bit string of fixed size using a one-way function. In some embodiments, the application of the cryptographic hash function cannot be reversed without a brute-force search of all possible sequence inputs into the testing sequence database. In some embodiments, the application of the cryptographic hash function further includes use of one or more information keys that must be accessed to attempt the brute-force search. In certain embodiments, the application of the cryptographic hash function requires keys from a plurality of independent sources that must cooperate to compute the hash without any one server gaining access to the sequence data. In certain embodiments, the independent sources comprise independent computer servers. In certain embodiments, the method also includes dividing the prepared testing sequence database into two or more partial testing sequence databases, and the prepared testing sequence database used for detecting of the presence or absence of a sequence match is one two or more partial testing sequences databases. In some embodiments, if a sequence match is detected between the partial testing sequence database and one or more fragments of the test biological molecules, the method further includes detecting the presence or absence of a sequence using another of the two or more partial testing sequence databases. In some embodiments, the testing sequence database contains a portion of a larger database of sequence fragments such that the fragments included in the testing sequence database can be rotated frequently or upon a match being discovered. In some embodiments, the method also includes acting in response to the detecting, wherein the acting includes one of more of preventing synthesis of the test biological molecule, permitting synthesis of the test biological molecule, sequencing one or more polynucleotide molecules, DNA sequencing, DNA molecule design, determining an amino acid sequence of a polypeptide, and further sequence identification steps. In certain embodiments, if the presence of a sequence match is detected the action includes preventing synthesis of the test biological molecule. In some embodiments, the method also includes identifying in the prepared testing sequence database one or more sequence fragments of the preselected biological sequence that match one or more sequence fragments, respectively, of a second biological molecule having a biological function unrelated to the biological function of interest of the preselected biological molecule, and removing the identified sequence fragments(s) from the testing sequence database

In another aspect of the invention, a testing sequence database prepared by any embodiment of any of the aforementioned methods is provided.

In another aspect of the invention, a method of assessing a biological sequence using an embodiment of an aforementioned testing sequence database is provided. In certain embodiments, the assessing includes determining whether to permit or prevent synthesis of the assessed biological sequence.

### BRIEF DESCRIPTION OF THE DRAWINGS

FIG. 1 is a diagram depicting how nucleic acid and peptide sequences can be broken into pieces of a certain length in order to detect exact matches within a database of pieces unique to potential bioweapons. The database may include known sequence fragments from potential bioweapons and/or computed functional equivalents, but does not include fragments matching functionally unrelated sequences from public databases.

FIG. 2 is a diagram showing how sequences to be screened and database contents can be hashed to permit screening while avoiding providing any information in cleartext.

FIG. 3A-B provides a depiction of how to create a database of sequences corresponding to potential bioweapons for exact match comparison to nucleic acid sequences to be examined. FIG. 3A provides a schematic illustrating use of fragments of nucleic acids or peptides of the appropriate size compute functionally equivalent fragments in a list rank-ordered by probability of function. FIG. 3B shows items from a rank-ordered list and illustrates that items may be included up to a random adversarial threshold value in the database in a deterministic, random, or biased random manner.

FIG. 4A-B provides graphic representations of how randomly choosing fragments from potential bioweapons, optionally in a manner biased towards conserved regions, can be used to reliably detect sequences corresponding to functionally similar bioweapons. FIG. 4A depicts five fragments and computed functionally equivalent variants along with depictions of naive or sophisticated “attacks” that seek to evade detection by introducing mutations throughout the sequence of the bioweapon. FIG. 4B illustrates failure of attempts to evade screening because the adversary does not know which fragments or how many functional variants of those fragments are included in the database and, in an attempt to avoid rendering the function nonfunctional due to including too many mutations, guesses variants that are included in the database.

FIG. 5 depicts the number of false positives anticipated per year for estimated levels of global DNA synthesis over time, assuming a database size of approximately one billion fragments and nucleic acid sequences of 57 base pairs or peptide sequences of 19 amino acids.

FIG. 6 is a schematic diagram of a flowchart that provides an overview of an embodiment of the invention and illustrates use of an exemption list.

FIG. 7 shows a RAT screening diagram that illustrates how different fragment windows across a gene have different fitness costs when mutated: some can be changed to almost anything, others tolerate a few substitutions but otherwise break the function, while still others exhibit a gradient in which most individual mutations impose a small cost that increases as more mutations are added. SecureDNA is a screening database prepared using an embodiment of a method of the invention.

FIG. 8 provides a schematic diagram of an embodiment of a phagemid-based selection used to measure the fitness of sequence variants of M13, an example virus that infects E. coli. Enrichment/de-enrichment relative to wild-type corresponds to variant fitness.

FIG. 9 illustrates a series of experimental steps used to generate data on the fitness of sequence variants of M13 in an embodiment of the invention.

FIG. 10 is a graph showing the effect of repeated selection for extrusion and infection on the library of variants of the polypeptide PQSVECRPFVFGAGKPYEF of M13 pIII.

FIG. 11 provides an enrichment profile for all single mutants of the alanine at position 13 in the polypeptide PQSVECRPFVFGAGKPYEF (SEQ ID NO: 23) of M13 pIII. The results indicated that all nineteen mutations were tolerated at this position.

FIG. 12 shows an enrichment profile for all single mutants of the proline at position 1 in the polypeptide PQSVECRPFVFGAGKPYEF (SEQ ID NO: 23) of M13 pIII. The results indicated that no mutations were tolerated at this position.

FIG. 13 is a chart that provides predictions obtained using the funtrp program for all amino acids in the polypeptide PQSV funtrp analysis. Column N corresponds to the likelihood that the position is neutral and accepts most or all substitutions with minimal loss of function. Column R corresponds to the likelihood that the position is a rheostat that accepts some number of mutations that reduce the function to varying degrees. Column T corresponds to the likelihood that the position is a toggle that does not tolerate mutations without losing function.

FIG. 14 provides a graph showing the Receiver Operating Characteristic (ROC) curve for a weak classifier based on the tools FUNTRP and BLOSUM62 as assessed against biological ground truth data obtained in a laboratory. The relevant data is for the sequence fragment window PQSVECRPFVFGAGKPYEF (SEQ ID NO: 23) from the pIII protein of M13, a filamentous virus that infects E. coli as described in Example 7. The selection used is depicted in FIG. 8, with the enrichment/de-enrichment scores compared for NGS point 1 and NGS point 6. The curve demonstrates that for the range of sequences tested, 90% of true positives could be predicted at a cost of 50% false positives.

FIG. 15 provides a graph showing another Receiver Operating Characteristic (ROC) curve for the same classifier (as in FIG. 14) with the enrichment/de-enrichment scores compared for NGS point 1 and NGS point 4. That the ROC curve is similar demonstrates that repeated selections were not necessary.

### DETAILED DESCRIPTION

Aspects of the invention, in part, include methods and systems with which to reliably and efficiently detect sequences corresponding to a preselected biological function, also referred to herein as “functional sequences” while minimizing the detection of functionally unrelated sequences, also referred to herein as: “unrelated sequences”. In some embodiments, methods of the invention include detecting nucleic acid functional sequences. In some embodiments, methods of the invention include detecting polypeptide functional sequences. As used herein the term “polypeptide” is used interchangeably with the term “protein”. An embodiment of a detection system of the invention may comprise a testing sequence database as described herein.

Methods of the invention can be used to detect nucleic acid or peptide sequences corresponding to a particular critical biological function such that sequences encoding that function can be reliably identified with a minimal chance of incorrectly identifying sequences that do not correspond to that function.

Certain embodiments of the invention are useful for preventing one interested in a sequence considered undesirable (also referred to herein as an “adversary”) for synthesis to avoid detection. Randomly choosing the fragments from the functional sequence prevents adversaries from knowing which fragments will be screened, forcing the adversary to include mutations throughout the entire test sequence in an attempt to evade detection. If the adversary does not include enough mutations at a particular fragment, their sequence may match one of the computed functional variants included in a testing sequence database of the invention. The more fragments included, and the more computed functional variants of those fragments, the greater the likelihood of detection. If the adversary includes too many mutations throughout their test sequence, it will no longer perform the desired function [Gray et al. Genetics 207 (1): 53-61 (2017); Jackson et al. PloS One 12 (4): e0164905 (2017) and Pokusaeva et al. PLoS Genetics 15 (4): e1008079 (2019)].

### Testing Sequence Database

In some embodiments of the invention, detection methods comprise searching for and/or identifying sequence matches to a database of sequences. In some embodiments, a database of sequences, also referred to herein as a “testing sequence database” comprises a plurality of sequence fragments of a preselected biological molecule. In some embodiments, a preselected biological molecule is selected at least in part, because it is capable of a function of interest. A preselected biological molecule may be a polypeptide molecule or may be a polynucleotide molecule and the preselected biological molecule may be capable of a function of interest. Non-limiting examples of a biological molecule capable of a function of interest include: a sequence corresponding to a virus capable of human-to-human transmission, such as, but not limited to Ebolavirus; and a sequence encoding a toxin capable of killing mammalian cells at very low doses, such as, but not limited to ricin. Additional biological molecules capable of a function of interest are known in the art and such sequences may be included in embodiments of methods of the invention.

A testing sequence database may be prepared in a manner such that it comprises a plurality of sequence fragments of the sequence of the preselected biological molecule, such fragments may also be referred to herein as “preselected sequence fragments.” In some embodiments of the invention, preselected sequence fragments in a testing sequence database are of a predetermined length. In embodiments in which a preselected biological molecule is a polynucleotide, a predetermined length of a preselected sequence fragment is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50, 55, 60, 65, 70, 75, 80, 85, 90, 95, 100, 110, 120, 130, 140, 150, 160, 170, 180, 190, 200, 300, or more nucleotides, including all integers between 15 and 300. In embodiments in which a preselected biological molecule is a polypeptide, a predetermined length of a preselected sequence fragment is: 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 91, 2, 93, 94, 95, 100, 110, 120, 130, 140, 150 or more amino acids, including all integers between 7 and 150. In certain embodiments, a testing sequence database includes preselected sequence fragments of the same predetermined length. In some embodiments, a testing sequence database includes preselected sequence fragments of different predetermined lengths.

In certain embodiments of the invention, a plurality of sequence fragments of the preselected biological molecule includes all or a significant portion of possible fragments of the biological molecule capable of the function of interest. In certain embodiments of methods of the invention, a plurality of sequence fragments of the preselected biological molecule does not include sequences that are found in a biological molecule capable of a function unrelated to the function of the preselected biological molecule. As used herein the term “plurality” means more than one, for example, it may mean at least 2, 3, 4, 5, 6, 7, 8, 9, 10, or more.

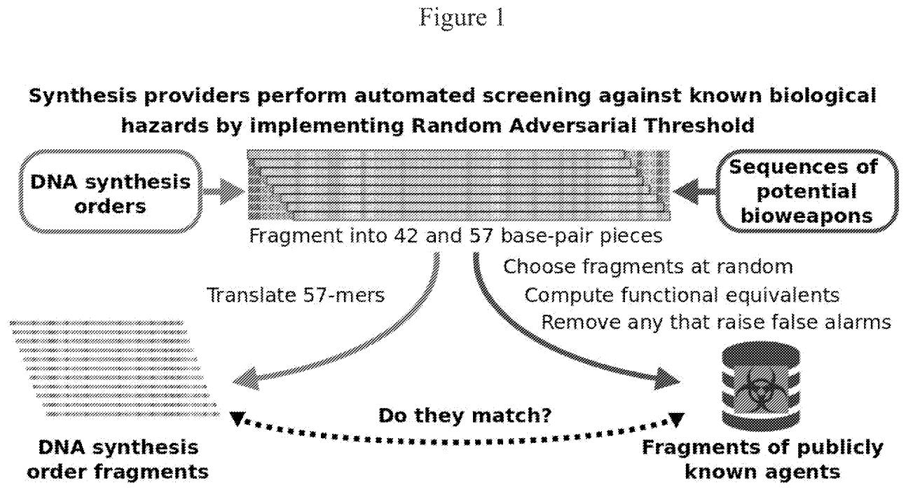

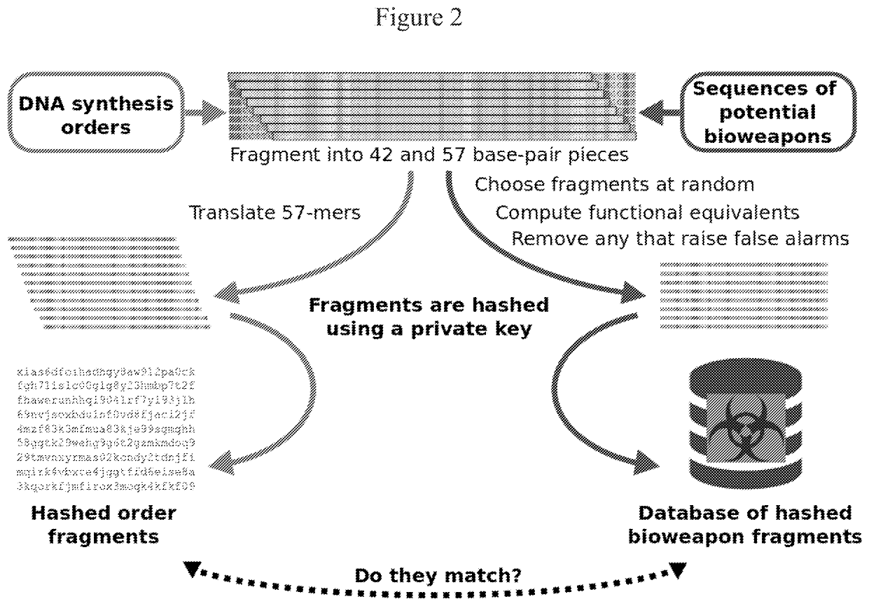

FIGS. 1 and 2 provide diagrams depicting certain embodiments of methods of the invention. FIG. 1 illustrates a way nucleic acid and peptide sequences can be broken into pieces of a certain length in order to detect exact matches within a database of pieces unique to potential bioweapons. In some embodiments of the invention, the database may include known sequence fragments from potential bioweapons and/or computed functional equivalents, but does not include fragments matching functionally unrelated sequences from public databases. FIG. 2 shows how in certain embodiments of the invention sequences to be screened and database contents are hashed to permit screening while avoiding providing any information in cleartext.

### Certain Means for Preparing a Testing Sequence Database

In some embodiments of the invention, a means of preparing a testing sequence database comprises includes screening the plurality of sequence fragments of a preselected biological sequence molecule against at least one control sequence database, wherein the control sequence database comprises a plurality of control sequence fragments of at least one molecule capable of a function of interest that is a function unrelated to the function of interest of the preselected biological molecule. The means of preparing the testing sequence database may also include: identifying the presence of a match between a sequence fragment in the plurality of sequence fragments of the preselected biological molecule and a sequence fragment in the control sequence database that is a fragment of the biological molecule identified as capable of a function unrelated to the function of interest of the preselected biological molecule. As used herein, the term “control sequence database” means a database that includes one or more groups of sequences unrelated to those being sought by the detection system whose inclusion in the testing sequence database would lead to a false positive match. Non-limiting examples include GenBank, the European Nucleotide Archive, and the sequences of all plasmids in the Addgene repository that have been requested by at least 25 laboratories.

Further, a means of preparing a testing sequence database may also include removing from the testing sequence database one or more sequence fragments of the preselected biological sequence identified as matching a sequence fragment of the biological sequence identified as capable of a function of interest unrelated to the function of interest of the molecule capable of the function of interest. As used herein the term “biological sequence” refers to a molecule found in a biological system, non-limiting examples of a biological sequence are a DNA sequence, an RNA sequence, a gene sequence, a polynucleotide sequence, a protein sequence, a polypeptide sequence, an amino acid sequence, and a nucleic acid sequence.

In some embodiments of methods and systems of the invention, a testing sequence database comprises randomly chosen fragments of functional sequences. In some instances, a rank-ordered list of sequences predicted to be functionally equivalent to sequences to include in a testing sequence database are computed using art-known methods, (see for example: Bromberg, Y., & B. Rost Nucleic Acids Res. 35, 3823-3835 (2017); Miller et al. Sci. Rep.7, 41329 (2017); Miller, M. et al. Nucleic Acids Res. 47, e142 (2019); Choi, Y. et al. PLoS One 7(10), e46688 (2012); Hopf, T. A. et al. Nat. Biotechnol. 35, 128-135 (2017); Gray, V. E. et al. Cell Syst. 6, 116-124.e3 (2018); and Riesselman, A. J. et al. Nat. Methods 15, 816-822 (2018), the contents of each of which is incorporated herein by referenced in its entirety]. A minimum number, non-limiting examples of which are: 1, 2, 3, 4, 5, 6, 7, 8, 9, 10, or more of the list of functionally equivalent sequences may be included in the testing sequence database for detection purposes. For example, such functionally equivalent sequences may be included as control sequences.

In some embodiments of methods of the invention, one or more computed equivalently functional sequences may be chosen at random or in a biased random manner from a rank-ordered list of sequences predicted to be functionally equivalent are computed using art-known methods and are included in a testing sequence database of the invention. In some embodiments, randomly chosen fragments and computed equivalently functional fragments are pre-screened for matches to known unrelated sequences present in databases, a non-limiting example of which is GenBank, to ensure that fragments that would falsely implicate an unrelated sequence are not included in the testing sequence database.

Some embodiments of the invention include a prescreening step in which sequences unrelated to a sequence of a preselected biological molecule are tested. In some embodiments of the invention, a rate at which unrelated sequences from the set of known sequences included in a pre-screening step before populating the testing sequence database are incorrectly identified is 0%. The rate at which unrelated sequences not known or not included in a pre-screening step are incorrectly identified by random chance varies with the length of fragments and the number of fragments included in the database, with the incorrect identification rate per fragment corresponding to one per the total number of nucleic acid or peptide sequences of the defined length. Use of devices, systems, and methods of the invention may reliably identify true functional sequences at rates of 90%, 95%, 99%, 99.9%, or 100%, including all percentages in the range provided, with the exact rate dependent upon the number of randomly chosen fragments and equivalently functional sequence fragments included in the testing sequence database.

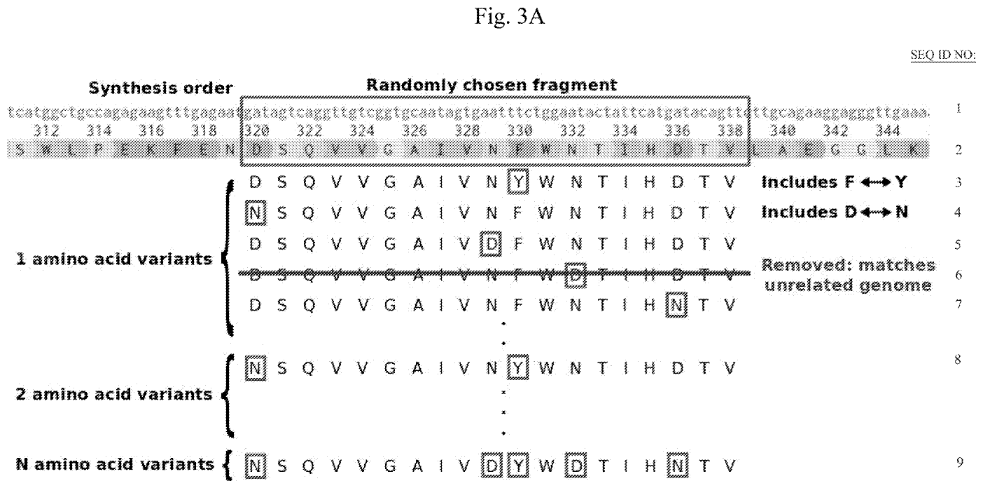

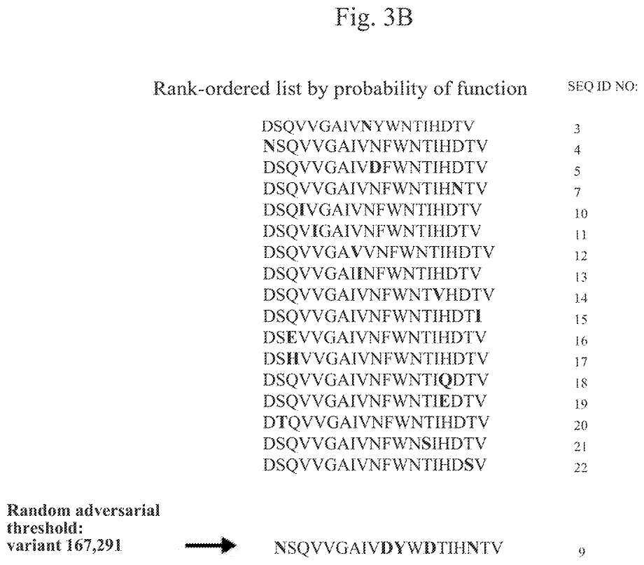

FIG. 3A-B illustrates a non-limiting example of preparing a database of the invention. In this example, a database of sequences corresponding to potential bioweapons is prepared for exact match comparison to nucleic acid sequences to be examined. FIG. 3A provides a schematic diagram that shows the use of fragments of nucleic acids or peptides of a predetermined size to compute functionally equivalent fragments in a list rank-ordered by probability of function. FIG. 3B shows items from such a rank-ordered list and illustrates that items may be included up to a random adversarial threshold value in the database in a deterministic, random, pseudorandom, or biased random manner.

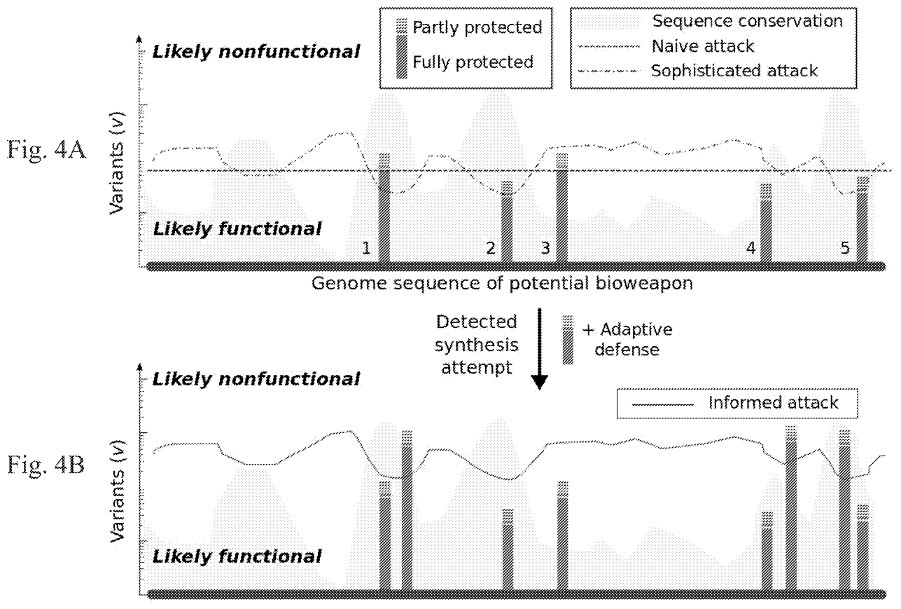

In another illustration of an embodiment of the invention, FIG. 4A-B presents graphic representations showing how the random choice of fragments from potential bioweapons, optionally in a manner biased towards conserved regions, can be used to reliably detect sequences corresponding to functionally similar bioweapons. As used herein the term “biased towards conserved regions” refers to the choice of sequences exhibiting a higher level of homology with the sequences of related genes and organisms. Such homology is often associated with greater functional importance. FIG. 4A depicts five fragments and computed functionally equivalent variants along with depictions of naive or sophisticated “attacks” that seek to evade detection by introducing mutations throughout the sequence of the bioweapon. FIG. 4B illustrates failure of attempts to evade screening because the adversary does not know which fragments or how many functional equivalents (also referred to herein as functional variants) of those fragments are included in the database and, in an attempt to avoid rendering the function nonfunctional due to including too many mutations, guesses variants that are included in the database. The term “functional equivalent” as used herein in reference to a first nucleotide or polypeptide subsequence means a second nucleotide or polypeptide subsequence, respectively, that can be substituted for the first nucleotide or polypeptide subsequence without imposing a substantial cost to the function of the overall sequence, biomolecule, or molecule encoded by the sequence.

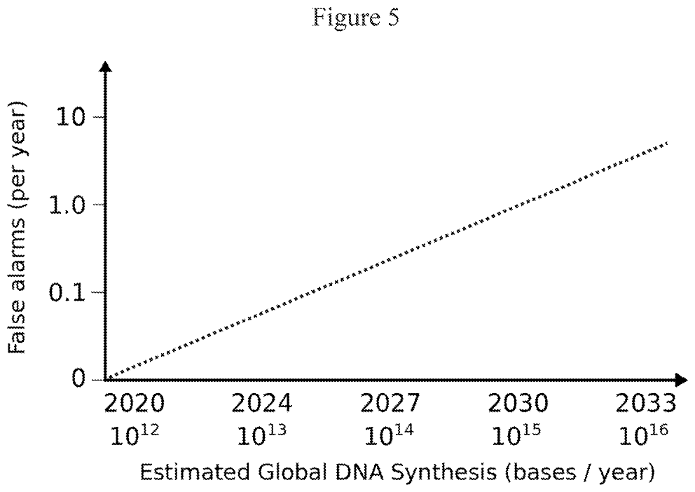

A database prepared using an embodiment of a method of the invention is expected to permit identification and provide an opportunity to prevent production of potentially hazardous nucleotide and/or polypeptide sequences. When screening sequences using a database prepared using an embodiment of the invention likelihood of false positive results is quite low. For example, FIG. 5 depicts the number of false positives anticipated per year for estimated levels of global DNA synthesis over time, assuming a database size of approximately one billion fragments and nucleic acid sequences of 57 base pairs or peptide sequences of 19 amino acids.

### Exemption Lists

The terms “exemption sequence” and “exemption list” are used herein in reference to sequences an individual and/or laboratory is explicitly authorized to use. Thus, when requesting synthesis of a sequence, the individual or laboratory requesting the sequence may provide the synthesis facility with a list of sequences the individual and/or laboratory is permitted to have and/or use. As a non-limiting example, a laboratory may be permitted to work with sequence “X”, which is considered a hazardous sequence but necessary for the lab to use in research to develop treatments or vaccines to an organism comprising sequence “X”. Other individuals and/or laboratories would not be permitted to synthesize or use sequence “X” but it would be considered an exemption sequence for the permitted laboratory, and would be on the laboratory's exemption list.

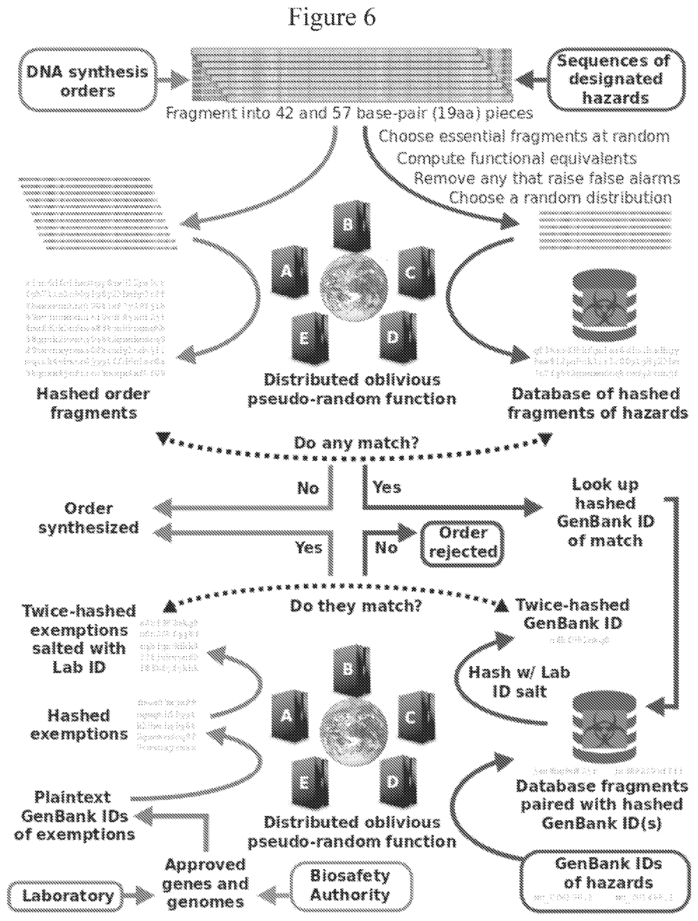

FIG. 6 provides a flowchart showing an overview of an embodiment of the invention and illustrates how an exemption list works. For example, laboratories are typically required to obtain permission to work with certain agents from their institutional biosafety committee or other authority. Certain embodiments of a fully automated screening system prepared using methods of the invention would recognize that such laboratories are allowed to obtain DNA corresponding to genes and genomes that they are permitted to work with, without requiring any human intervention. In a non-limiting example, each gene and genome listed in a biosafety committee authorization report has an associated GenBank® ID, because all genes and genomes do. An exemption list comprises all GenBank IDs of genes and genomes that the lab is explicitly authorized to use. This information can be used to identify permitted genes and genomes to a screening system of the invention. In some embodiments of a system of the invention, each laboratory that wants sequences synthesized is required to send their exemption list to the screening system of the invention, which hashes each GenBank ID once using the distributed oblivious multiparty server system, then hashes it again using the laboratory's unique ID as a salt. This ensures that all laboratories have different and unique hashes, keeping the lists private and preventing an adversary with a copy of the database and knowledge of which laboratories work on which genes and genomes from determining which hash corresponds to which gene or genome. Thus, an adversary is prevented from using this information to determine what is present in the database. When hazardous genes or genomes are entered into the database to be protected by operators of the screening system of the invention, the corresponding GenBank ID is hashed once using the distributed oblivious multiparty server system and associated with each hashed sequence fragment of that gene or genome that is in the database. If a user places an order for which the system of the invention detects that a DNA fragment is present in the hazard database, the database hashes the associated (once-hashed) GenBank® ID using the customer's laboratory ID, then checks to see if the resulting hash matches a (similarly hashed) entry on the customer's exemption list. If so, the order goes through and the requested sequences are synthesized. If not, the system of the invention rejects the order and may record the incident.

### Fitness Costs

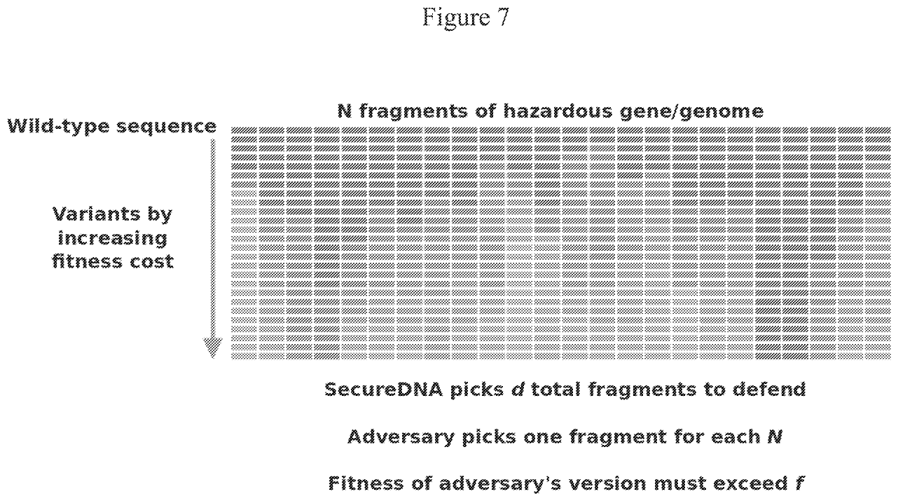

In some embodiments of method of the invention, include assessing how fragments of a sequence of a gene or gene product have different fitness costs when mutated. FIG. 7 shows a RAT screening diagram that illustrates how some fragment windows across a gene can be changed to almost anything, other fragment windows will tolerate a few substitutions but otherwise the substitutions break the gene or gene product's function, while still other fragment windows of a sequence exhibit a gradient in which most individual mutations impose a small cost that increases as more mutations are added. The random adversarial threshold approach described herein with respect to methods of the invention works by using a classifier algorithm to predict the function of mutants for each fragment window and including many of the wild-type sequences and predicted mutant sequences in the database. To obtain a functional version of a hazardous gene or genome protected by the system of the invention, an adversary must choose to place a synthesis order with either the wild-type sequence or a variant for each window of the gene or genome. For the order to go through, all these guesses must not be present in the database. Because the adversary does not know which windows are protected, they must guess at a variant for every window, incurring a potential fitness cost and risk of discovery at each window. For the resulting sequence to be functional, the total cost of the system must not exceed a minimum threshold. The greater the fraction of variants protected within a given window and the more windows protected, the lower the likelihood that the order will escape detection. Because the approach can be fully automated, it can function for next-generation desktop DNA synthesizers as well as central providers.

### Certain Applications of Methods

Certain embodiments of the invention are useful for biosecurity applications. For example, though not intended to be limiting methods of the invention can be used to detect functional sequences corresponding to bioweapons in DNA synthesis orders in order to prevent such synthesis and reject those orders. Another non-limiting implementation of an embodiment of a method of the invention includes detecting functional sequences corresponding to bioweapons in DNA sequencing results. Another non-limiting implementation of an embodiment of the invention includes detecting functional sequences corresponding to bioweapons from a set of sequences entered into DNA design and analysis software programs.

Certain aspects of the invention permit highly efficient computation of whether a sequence is functional. Times corresponding to O(log(N)) are considered the gold standard for an optimally fast algorithm, where in the context of the invention N corresponds to the number of fragments in the database (Cormen, T. H., et al., 2009. Introduction to Algorithms. MIT Press.) Some data structures permit exact-match lookup with times corresponding to 0(1); because the invention relies on exact-match lookup, certain embodiments of the invention permit similar efficiencies.

Certain embodiments of the invention permit automated screening for functional sequences without human intervention. For example, according to embodiments of methods of the invention a nucleic acid synthesis or peptide synthesis machine may be programmed to automatically screen for and reject sequence synthesis orders that include a functional sequence derived from proscribed list of organisms and toxin genes, such as those from the U.S. Federal Select Agent Program (F SAP) and the Australia Group treaty for harmonized export control.

### Molecules and Sequences for Screening

In some embodiments of the invention, a test sequence is screened against a testing sequence database in a method or system of the invention. The term “screened against” means “compared with.” In a non-limiting example a sequence of interest to synthesize is a test sequence and it is screened using a using a method and/or system of the invention. The screening against a testing sequence database of the invention provides information that can assist in determining an action to be taken with respect to the test sequence, such as but not limited to: whether to permit or prevent the sequence to be synthesized. Other actions that may be informed by results of applying embodiment of a method of the invention to a test sequence include, but are not limited to sequencing one or more polynucleotide molecules, DNA sequencing, DNA molecule design, further sequence identification steps. Various means of assessing DNA molecule design, sequencing of DNA and/or protein sequences are known in the art and can be applied as part of an action taken based at least in part on information resulting from use of an embodiment of a testing database of the invention.

In some embodiments of the invention, a test biological molecule is fragmented into one or more of: a plurality of, some of, and all possible overlapping pieces shifted by one base pair or one amino acid of the desired length for comparison to equivalently sized pieces of related sequences. The fragmented sequences of the one or more test biological molecules are of lengths equivalent to a predetermined length of the sequence fragments of the preselected biological molecule in the testing sequence database. A test biological molecule is a molecule that is assessed/tested using a testing sequence database of the invention. For example, although not intended to be limiting, a test biological molecule may be a polynucleotide that an individual or lab wants to synthesize or have produced by a service provider or synthesizer.

### Sequence Identity Protection

In some methods and systems of the invention, the identity of each sequence fragment of one or both of the testing sequence database and the test biological molecule are protected. The term “protected” as used herein, means a user of a method of system of the invention is prevented from identifying the sequence of the fragment or the test biological molecule. For example, the sequence fragments to be screened can be “hashed” using methods known to those of the art to produce one-to-one information mappings that cannot be readily reversed. Including equivalently hashed fragments from related sequences in a testing sequence database permits reliable database lookup and detection without disclosing the identities of the sequences. Various art-known means of protecting the identity of sequences may be used. A non-limiting example of a means of protecting comprises application of a cryptographic hash function, wherein the cryptographic hash function deterministically maps the sequence data to a bit string of fixed size using a one-way function (see for example: Cormen, T. H., et al., 2009. Introduction to Algorithms. MIT Press.) Cryptographic hash functions are used in the art and art-known methods can be used to include cryptographic hash functions in methods and systems of the invention. In some embodiments of the invention a cryptographic hash function is selected and applied and cannot be reversed or deciphered without a brute-force search of all possible sequence inputs into the testing sequence database. In some embodiments of the invention, a cryptographic function applied also includes use of one or more information keys that must be accessed to attempt the brute-force search. The inclusion of such an information key or keys restricts the ability of a user to access the identity of each sequence fragment of one or both of the testing sequence database and the test biological molecule. It will be understood that additional means for protecting the identity of sequence fragments can also be used in conjunction with embodiments of methods of the invention. See for example: Yao, A. C. 27th Annual Symposium on Foundations of Computer Science (sfcs 1986), Toronto, ON, Canada, 1986, pp. 162-167 and I. Damgård, I. ′89 Proceedings, Lecture Notes in Computer Science Vol. 435, G. Brassard, ed, Springer-Verlag, 1990, pp. 416-427, the content of each of which is incorporated by reference herein in its entirety.

### EXAMPLES

### Example 1

A testing sequence database is constructed by choosing all possible fragments from proscribed lists of organisms and toxin genes, such as those from the U.S. Federal Select Agent Program (FSAP) and the Australia Group treaty. The database is pre-screened against a known database such as GenBank to remove any that match functionally unrelated sequences.

A DNA synthesis provider fragments sequences from customer orders into all possible overlapping pieces equivalent in size to those in the database. Fragments are translated in all possible reading frames to produce equivalent peptides. The fragments from customer orders are compared to those in the database in an automated manner.

### Results

The synthesis provider is capable of screening all orders for fragments exactly matching those from proscribed lists, with few or no false positives corresponding to unrelated sequences. Screening can be done in a fully automated manner, avoiding the cost of human experts.

### Example 2

A testing sequence database is constructed by randomly choosing fragments, optionally biased towards highly conserved regions, from proscribed lists of organisms and toxin genes, such as those from the U.S. Federal Select Agent Program (FSAP) and the Australia Group treaty. Functional equivalents of these fragments are computed using predictive programs or algorithms and a random number are included in the database. The database is pre-screened against a known database such as GenBank to remove any fragments that match functionally unrelated sequences.

A DNA synthesis provider fragments sequences from customer orders into all possible overlapping pieces equivalent in size to those in the database. Fragments are translated in all possible reading frames to produce equivalent peptides. The fragments from customer orders are compared to those in the database in an automated manner to detect orders that would produce functional equivalents of proscribed organisms or toxin genes.

### Results

The synthesis provider is capable of screening all orders for fragments that are functionally equivalent to those from proscribed lists, with few or no false positives corresponding to functionally unrelated sequences. Screening can be done in a fully automated manner.

### Example 3

A testing sequence database is constructed by randomly choosing fragments, optionally biased towards highly conserved regions, from proscribed lists of organisms and toxin genes, such as those from the U.S. Federal Select Agent Program (FSAP) and the Australia Group treaty. Functional equivalents of these fragments are computed using predictive programs or algorithms and a random number are included in the database. The database is pre-screened against a known database such as GenBank to remove any fragments that match functionally unrelated sequences.

A DNA synthesis provider assigns an informational key to each customer interested in securing their orders against industrial espionage. Customer orders are fragmented into all possible overlapping pieces equivalent in size to those in the database, translated in all possible reading frames to produce equivalent peptides, and all results hashed using the key. The provider similarly hashes all sequences in the database. The fragments from customer orders are compared to those in the database in an automated manner to detect orders that would produce functional equivalents of proscribed organisms or toxin genes without sharing customer orders.

### Results

The synthesis provider is capable of screening all orders for fragments that are functionally equivalent to those from proscribed lists, with few or no false positives corresponding to functionally unrelated sequences. Screening can be done in a fully automated manner. Screening can be done in a fully automated manner without requiring customers to provide their orders to the synthesis provider in cleartext, protecting customers from industrial espionage.

### Example 4

A testing sequence database is constructed by randomly choosing fragments, optionally biased towards highly conserved regions, from proscribed lists of organisms and toxin genes, such as those from the U.S. Federal Select Agent Program (FSAP) and the Australia Group treaty. Functional equivalents of these fragments are computed using predictive programs or algorithms and a random number are included in the database. The database is pre-screened against a known database such as GenBank to remove any fragments that match functionally unrelated sequences.

A DNA sequencing provider fragments sequencing results from customer samples into all possible overlapping pieces equivalent in size to those in the database. Fragments are translated in all possible reading frames to produce equivalent peptides. The fragments from customer orders are compared to those in the database in an automated manner to detect customers with materials capable of producing functional equivalents of proscribed organisms or toxin genes.

### Results

The sequencing provider is capable of screening all sequencing results for fragments functionally equivalent to those from proscribed lists, with few or no false positives corresponding to functionally unrelated sequences. Screening can be done in a fully automated manner.

### Example 5

A testing sequence database is constructed by randomly choosing fragments, optionally biased towards highly conserved regions, from proscribed lists of organisms and toxin genes, such as those from the U.S. Federal Select Agent Program (F SAP) and the Australia Group treaty. Functional equivalents of these fragments are computed using predictive programs or algorithms and a random number are included in the database. The database is pre-screened against a known database such as GenBank to remove any fragments that match functionally unrelated sequences.

A DNA design software provider fragments sequences entered by customers into all possible overlapping pieces equivalent in size to those in the database. Fragments are translated in all possible reading frames to produce equivalent peptides. The fragments from customer orders are compared to those in the database in an automated manner to detect customers who might be inadvertently or deliberately designing engineered constructs with functions equivalent to proscribed organisms or toxin genes.

### Results

The design software provider is capable of screening all designs for fragments functionally equivalent to those from proscribed lists, with few or no false positives corresponding to functionally unrelated sequences. Screening can be done in a fully automated manner.

### Example 6

A study was performed using an embodiment of a sequence screening method of the invention. This experiment tested a random sample of 10,000 variants of the window PQSVECRPFVFGAGKPYEF (SEQ ID NO: 23) within the gene PIII of the M13 bacteriophage, which is an example virus that infects E. coli bacteria and is harmless to humans. M13 was used in the study as a representative virus. In this case, a library of variant sequences was generated, sequenced, and subjected to repeated rounds of infection to select for mutants that retained function. After each round of selection, the survivors were sequenced in order to quantify the changing frequency of each variant. The classifier used FUNTRP and BLOSUM62 to produce a fitness estimate in arbitrary units, while for the purpose of determining ground truth, the phage was considered fit enough to be “hazardous” if the ratio of its measured proportion of representation in the larger phage population before and after propagation in bacterial culture exceeded a certain bound. In other words, the classifier attempted to name elements of [symbol] _{func}^{w}, while the ground truth of [symbol] _{func}^{w} was established experimentally according to a fitness bound fₘᵢₙ.

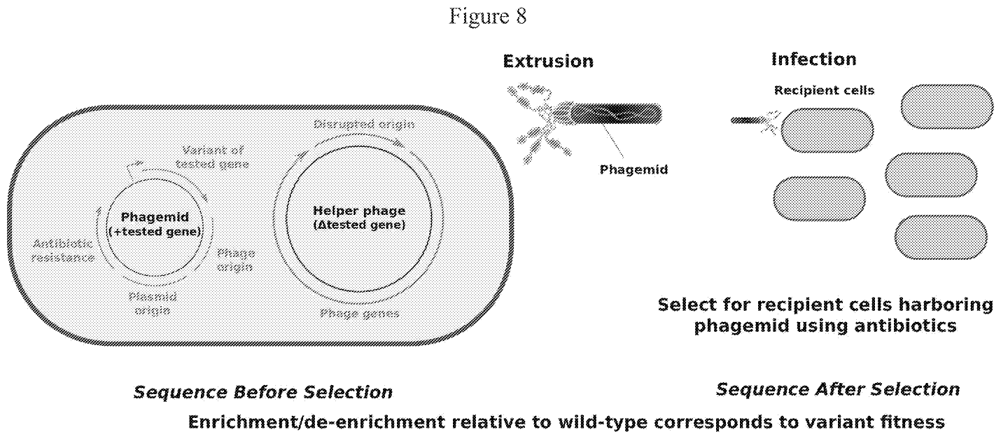

Experimental data on the effects of substituting variants of 19-amino acid windows within proteins and 42 base-pair sequences within functional nucleic acid sequences was obtained by evaluating the genome of the M13 virus that infects E. coli bacteria using the prediction tool funtrp (for protein sequences) and nucleic acid conservation (for the nucleic acid sequences in the viral replication origin and packaging signal). Fifteen stretches of 42- or 57-base pairs were identified for experimental investigation in the packaging signal, positive and negative replication origins, and genes I, II, III, and IV. An oligonucleotide library of 220,000 sequences comprising variants at positions predicted by funtrp or by structural analysis (for nucleic acids) was constructed to assess the accuracy of variant prediction. These libraries of variants were cloned into phagemids, which are plasmids with the M13 origin of replication and (for proteins) a copy of the relevant protein-coding gene from the M13 virus (see FIG. 8). Helper plasmids comprising the M13 phage with replication origins and packaging signal disrupted by insertion of the p15a plasmid origin and the kanamycin resistance gene were constructed. Each helper plasmid had all protein-coding genes intact (for nucleic acid studies) or had either gene I, gene II, gene III, or gene IV deleted. For example, a helper plasmid with gene I deleted can be complemented by the phagemid library encoding gene I variants to produce M13 particles encoding the phagemid library rather than the helper plasmid. Mixing these with recipient E. coli cells and selecting for successfully infected recipient cells carrying a phagemid effectively selects for phagemids that were able to complement the missing gene. The degree of enrichment or de-enrichment relative to the wild-type sequence corresponds to the fitness of the variant with respect to virus production and infection.

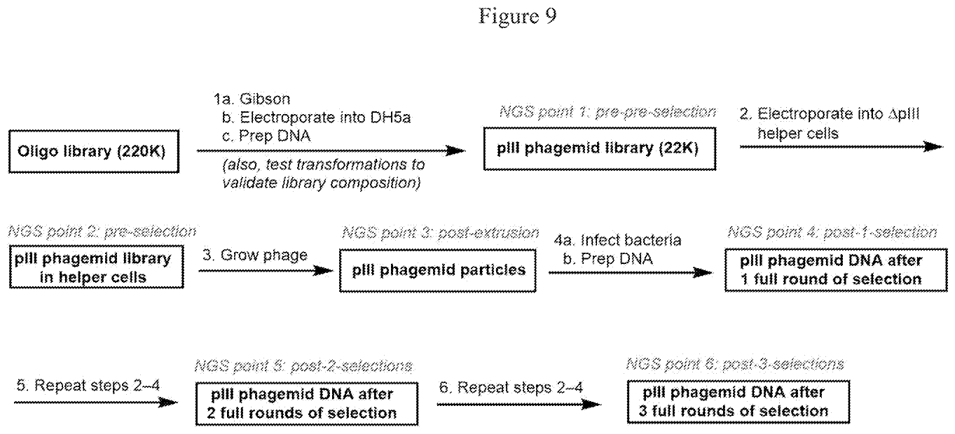

The library of gene III variants for the amino acid sequence PQSVECRPFVFGAGKPYEF (SEQ ID NO: 23) were initially cloned in DH5alpha cells and sequenced by MiSeq (see FIG. 9) to measure the initial library diversity (NGS point 1, initial library). They were then transformed into cells carrying the helper plasmid missing gene III, and sequenced again (NGS point 2, pre-selection). The resulting cells were grown up and M13 particles purified and sequenced (NGS point 3, phage extrusion), then mixed with recipient cells carrying a different antibiotic resistance marker. The resulting cells were grown up to select for both phagemid and recipient markers and sequenced (NGS point 4, post-1-selection). The selection was repeated twice more to obtain additional enrichment data (NGS points 5 and 6, post-2-selection and post-3-selection). Library sequencing coverage was ˜40x, with 100% coverage of the two pre-selection samples.

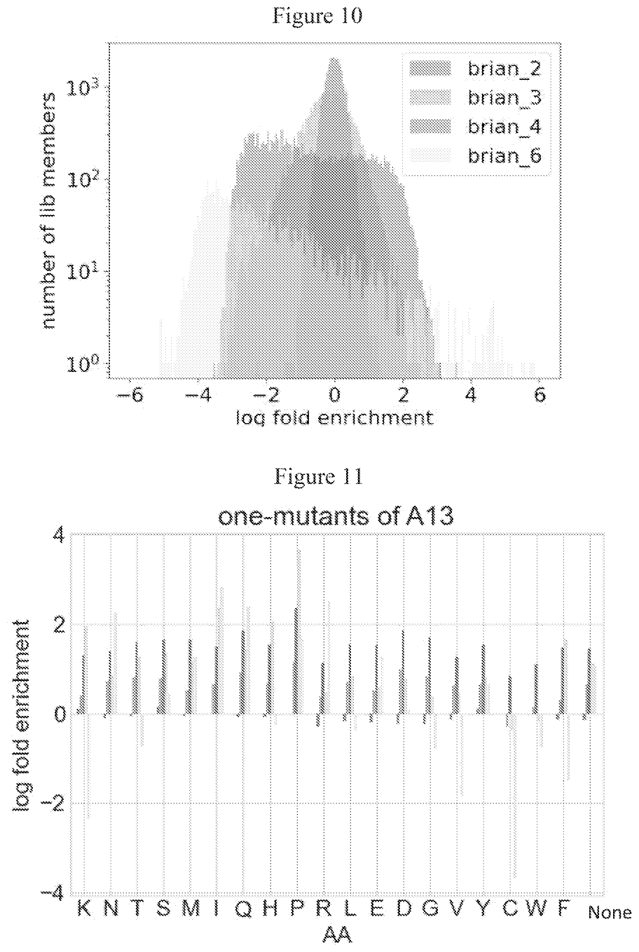

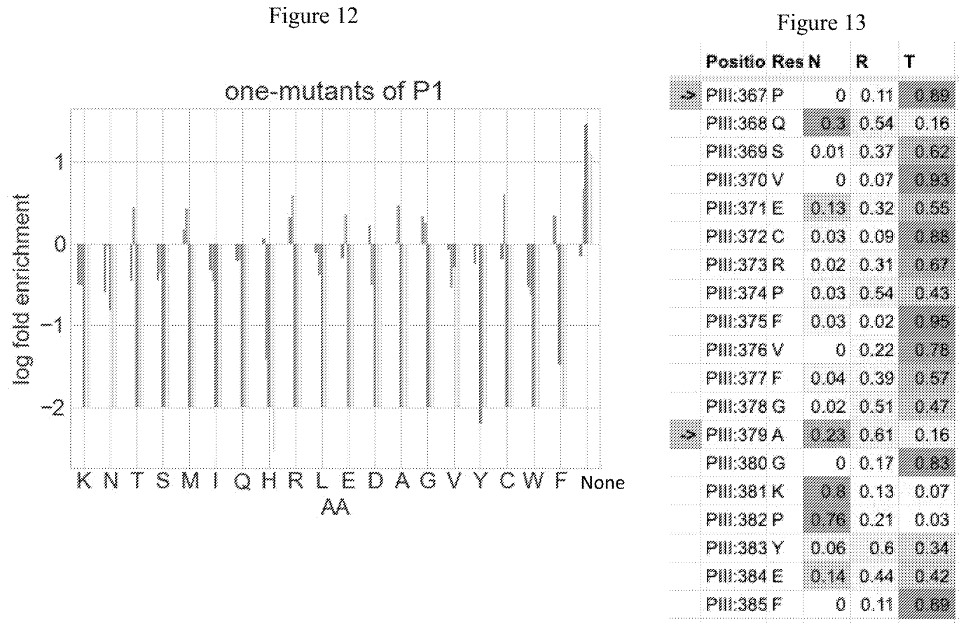

Selections led to enrichment/de-enrichment of four orders of magnitude in each direction (FIG. 10), indicating that the variants are indeed being selected by some criteria. Analysis of the fates of all one-mutant variants of particular amino acid residues revealed that the alanine at position 13 tolerates any mutation, while the proline at position one does not tolerate any (FIGS. 11 and 12).

Variants were considered fit if the ratio of their measured proportion of representation in the larger population before and after selection (NGS point 1 relative to NGS point 4 or point 6) exceeded a certain bound for a variety of threshold values. These distributions of fit and unfit sequences from the library were used as empirical datasets to evaluate prediction.

To predict function, funtrp analyses of how likely each position was to accept substitutions with zero, moderate, or high fitness cost (FIG. 13) were combined with a BLOSUM62 matrix defining common substitutions for the amino acids at those positions to generate predictions of functional variants for all variants in the library. These were compared to the empirical results, for a variety of threshold bounds, to establish ROC curves assessing the true positive and false positive rate for the combined funtrp+BLOSUM62 classifier. Notably, the ROC curves for NGS point 1 to point 4 and point 1 to point 6 were nearly identical, suggesting that only one round of selection is required.

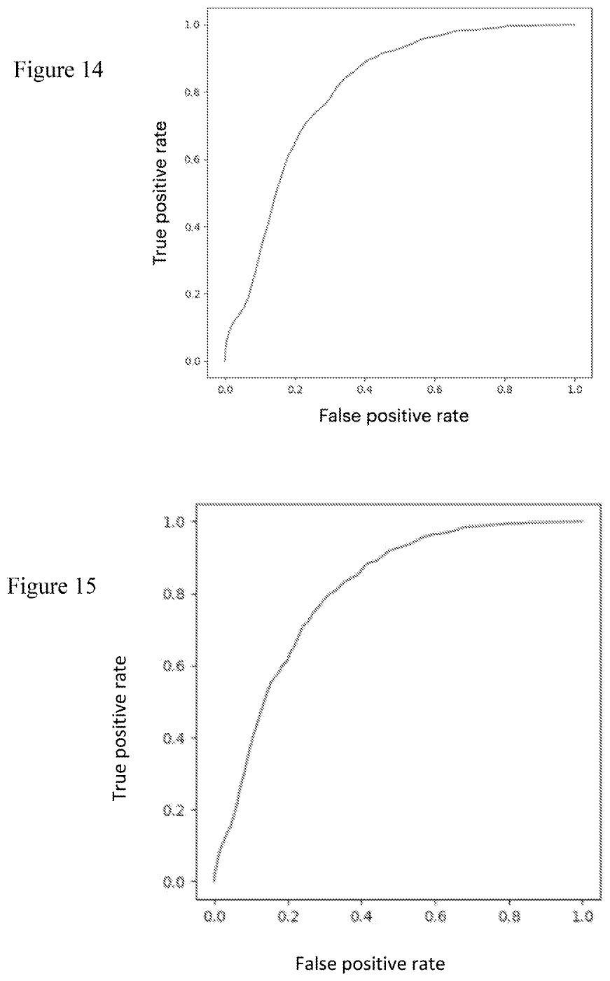

FIG. 14 shows a graph of an ROC curve generated from the experimental data. The graph is a receiver operating Characteristic (ROC) curve for a weak classifier based on the tools FUNTRP and BLOSUM62 as assessed against biological ground truth data obtained in the experimental study. The ROC curve captured the trade-off between Type I (false positive) and Type II (false negative) errors for a yes-no classifier. The false positive rate (horizontal axis) is the fraction of variants that were not classified as fit, and the true positive rate (vertical axis) is the fraction of fit variants that were correctly classified. The curve is parameterized by s E (−∞, ∞), the fitness threshold in arbitrary units that the classifier used to separate positives from negatives based on its noisy fitness estimate. FIG. 15 is a graph showing another Receiver Operating Characteristic (ROC) curve for the same classifier (as in FIG. 14) with the enrichment/de-enrichment scores compared for NGS point 1 and NGS point 4. That the ROC curve is similar demonstrates that repeated selections were not necessary.

Similar evaluations may be performed for other classifiers and for other variant libraries to improve prediction as needed. For nucleic acids, prediction may combine a conservation analysis of each position combined with a structural analysis calculating the change in folding energy of the relevant RNA secondary structure that occurs due to the mutation.

Notably, the ROC curve for funtrp+BLOSUM62 sufficed to predict 90% of functional sequences from the library at a cost of half the sequences being false positives. That is, given 10,000 functional sequences, the ROC curve indicates that the classifier could predict 18,000 and successfully cover 9,000 of the 10,000. If such sequences were included in the database at multiple positions across a hazard, the odds of detection become very high and the odds of the adversary obtaining a functional sequence given nondetection become quite low given the cost of including sufficient variants to have a chance at evading detection.

### Example 7

A testing sequence database is constructed by randomly choosing fragments, optionally biased towards highly conserved regions, from proscribed lists of organisms and toxin genes, such as those from the U.S. Federal Select Agent Program (FSAP) and the Australia Group treaty. Functional equivalents of these fragments are computed using predictive programs or algorithms and a random number are included in the database. The database is pre-screened against a known database such as GenBank to remove any fragments that match functionally unrelated sequences.

An adversary attempts to synthesize a functional version of a proscribed gene or genome. How can their odds of success or failure be determined?

The following describes an analysis of a screening method of the invention that was performed. The term “secureDNA system” refers to an embodiment of a screening method system of the invention. The system is used to identify sequences that are “hazardous” sequences and/or potential functional variants of hazardous sequences. In the description below, the term “individual” means an organism, such as a virus, bacteria, or other organism. Using the methods below, nucleotide sequences from an individual were assessed to determine if they were functional sequences, for example would, if included in the organism, permit the organism to survive and replicate. In this example, the term “adversary” means a person or entity to whom it is of interest to synthesize or to have synthesized a sequence that is considered a hazardous polynucleotide sequence. In the description below, the term “defender” means the operator of the system of the invention who seeks to prevent unauthorized persons and entities from synthesizing or otherwise accessing hazardous polynucleotide sequences.

The SecureDNA system succeeds in screening DNA if it prevents all adversaries from assembling sequences encoding functional biohazards. The most dangerous variety of hazard is a self-replicating agent capable of exponential spread without human assistance. A functional sequence for such a replicating agent is defined as a DNA sequence that has sufficient fitness to survive and replicate in the shared environment so as to become increasingly more common in the absence of human intervention, such as a novel pandemic virus. Fitness is formalized in a number of ways: the probability of a subject surviving to reproduce, the subject's expected number of offspring, or either of these normalized against some relevant population. In any case, a probability-like real number in [0,1] is a sufficient representation for fitness, and it can be assumed that there exists some minimum fitness fₘᵢₙ below which the agent dies out. If the maximum fitness for all hazard variants that can be synthesized despite SecureDNA is less than fₘᵢₙ, the SecureDNA system succeeds.

SecureDNA uses Random Adversarial Threshold (RAT) screening to search for fragments of hazards and plausibly functional variants. A variant is a DNA or amino acid sequence window that differs at one or more positions from the wild-type sequence (the sequence of a real agent one would find online, for example) at the same locus. Each hazard is composed of many loci, with any variant allowed at any window within any locus. The conditional distribution F(ν) was defined as the fitness, or functionality, of the hazard given variant ν, where ν is a triple (h, l, s_{ν}), h: hazard identity or index; 1: window index within genome; s_{ν}: exact variant sequence. The fitness of the wild type at any locus, F((h, l, s_{h:l})), was 1 by definition. Because sequence variants s_{ν} are typically unique to both the hazard and the window whenever F((h, l,s_{ν}))>∈, with slight abuse of notation, it could be said F (s_{ν})=F(ν). The total number of windows across the coding sequence of the hazard were indicated as N. Complex interactions between variants were possible, but it was assumed at least multiplicative compounding among small fitness adjustments from wild type, i.e., the fitness of a hazard with multiple variants was at most the product of the individual variants' fitnesses. An individual working with the SecureDNA system privately selects windows to screen within each hazard and the variants to be included using predictive software available in the art. Experiments were conducted using funtrp [Miller, M., et al., Nucleic Acids Research, 2019, Vol. 47, No. 21, e142] in combination with the BLOSUM62 matrix of amino acid substitution probabilities [Eddy, S. Nat Biotechnol 22, 1035-1036 (2004)].

For a given RAT database [symbol] , the adversary's task is to select a set of variants V such that [symbol] ∩ [symbol] is empty, and

$${\prod }_{v ϵ V} F ( v ) > {f}_{min}$$

which constitutes a failure of SecureDNA. In this example, it was conservatively assumed that the adversary has an oracle capable of perfectly predicting the fitness of any given variant, i.e., the adversary knows F(ν). It was noted that currently available methods of estimating F(ν) are extremely poor, so the information presented also includes some interpretation of the effect of significant inaccuracy in this estimate, which is a realistic condition for the assessment.

### 2. Breaking Changes Approximation

Actual fitness distributions can only be measured empirically and in part; a given experiment will struggle to assess more than a few million variants at a single window, and then only for biomolecules amenable to measurement. The study permitted a rough estimation of the fitness distribution for the most essential and evolutionarily conserved windows: for example, a sequence may tolerate substitutions with no more than a moderate fitness cost at nine out of nineteen amino acid residues, with seven, seven, five, five, four, three, three, one, and one alternative residues permitted, for a total of 737,280 variants that do not completely break the function of the hazard at that particular window F(ν)>0.5. This situation was conservatively approximated by assigning all of these a value of 1, and all remaining variants a value of 0.

The “breaking changes” approximation F_{b}(ν) was introduced for the fitness distribution as

$$F ( v ) \approx {F}_{b} ( v ) = \{ \begin{matrix}1 - ∈ & v ∈ {𝕍}_{func} \\ 0 & v ∉ {𝕍}_{func}\end{matrix}$$

where [symbol] _{func}, was the set of variants that were approximately as functional as the wild-type sequence. This approximation was good, e.g., when one amino acid served a critical topological or affinity role in its protein, so that only a small set of replacements would yield a functional protein and hazard. It was assumed that an individual choosing critical regions to screen was able to satisfy the conditions for this approximation.

The system included choosing k different windows, that is, k indices w into the hazard genome. Let V^{w} be the set of all variants (h, w, s_{ν}). The subset of all variants at this window that are functional is [symbol] _{func}^{w}. The coverage of a RAT database [symbol] at window w is

$${α}_{w} = \frac{\vert 𝔻 ⋂ {𝕍}_{func}^{w} \vert}{\vert {𝕍}_{func}^{w} \vert}$$

Assuming no preference between functional variants, which are assumed to all have effectively equal fitness, the probability of an adversary randomly choosing functional variants not present in the database at all k locations, an “evasion” event, called “E,” is

$$P ( E ) = {\prod }_{w ∈ \{ k \}} 1 - {α}_{w} \qquad ( 1 )$$

A notable bound on the probability of evasion is

$$P ( E ) \leq {( 1 - \frac{{\sum }_{w ∈ [ k ]} {α}_{w}}{k} )}^{k} \qquad ( 2 )$$

from the arithmetic-geometric mean inequality. More coarsely, a bound based could also be introduced on the maximum coverage αₘₐₓ, over all k windows:

P(E)≤1−αₘₐₓ (3)

In the case that we can establish even one strong guarantee on coverage, from 3, we can rely on the maximum coverage provided by any one window to bound P(E). A defender with perfect knowledge of F_{b}(ν) matching the adversary can potentially cover one or more windows with the lowest | [symbol] _{func}^{w}| completely, achieving the perfect defense of αₘₐₓ=1 and P(E)=0.

However, defenders do not have perfect knowledge of F_{b}(ν). From the defender's perspective, prediction of which variants are and are not members of [symbol] _{func} is uncertain, and even the degree of uncertainty of such a prediction is challenging to estimate. In the case in which only weak guarantees can be established on the average coverage, it can be seen from (2) that a means by which to compensate is to include more windows, i.e., increasing k.

A stronger bound, that also exploits that the location of the k windows is unknown to the adversary, is explored in Section 4.1, below herein. First, the following discussion relates to the effects of uncertainty in the defender's estimation of F_{b}(ν) assuming that the defender's choice of k windows is known to the adversary.

### 3. Error Trade-Offs

Suppose that prediction is a trade-off between Type I and Type II error, such that additional entries for each w are increasingly likely to be false positives as the defender attempts to cover a greater fraction of [symbol] _{func}^{w} using a noisy classifier. Such a trade-off is summarized by the classifier's receiver operating characteristic (ROC) curve, traditionally given by

$$\{ \frac{{tp}_{w} ( s )}{{fp}_{w} ( s )} s ∈ ( - ∞ , ∞ )$$

where tp_{w}(s) and fp_{w}(s) are the true positive and false positive rates, respectively, of identifying a functional variant, i.e., distinguishing a member of [symbol] _{func}^{w}, and s is a threshold parameter dictating how aggressively we include potentially functional variants.

The ROC curve precisely captures the trade-off between Type I and Type II errors. Choosing a point on the curve, based on a selection criterion and referred to as the operating point, constitutes a specific compromise, which can be selected in a principled way.

There are many ways to quantify and optimize over an ROC curve. One useful example is to define costs Cₜₚ, C_{fp} C_{fn}, and Cₜₙ as the costs of true positive, false positive, false negative, and true negative test outcomes, respectively, in a game theoretic sense. Then, assuming a convex and differentiable ROC curve (generally resulting from a fit to data), a unique optimal point on the curve at s=s_{w,opt} may be selected based on a tangency criterion [see England, W.L., Medical Decision Making, 1988 vol. 8(2):120-131, the content of which is incorporated herein by reference in its entirety.]

$$\frac{dtp}{dfp} = \frac{1 - q}{q} \frac{{C}_{tn} - {C}_{fp}}{{C}_{tp} - {C}_{fn}} \qquad ( 4 )$$

where q is the “base rate” of functional sequences in the subset of sequence space considered. It is certainly true that

$$q \geq \frac{\vert {𝕍}_{func} \vert}{\vert 𝕊 \vert}$$

though this is an almost vacuous lower bound given the huge size of | [symbol] | relative to the number of functional sequences. In reality, no adversary or defender would choose variants outside of a certain Hamming distance r before the variants become too different from the wild type to ever function. r is an empirical biological parameter. One potential expression for q might be

$$q = \frac{\vert {𝕍}_{func} \vert}{H ( 𝕊 , r )} \qquad ( 5 )$$

Where H ( [symbol] , r) is the volume of a Hamming ball of radius r within the set [symbol] . This Hamming ball volume may be understood as size of the set of reasonable variants that could conceivably be functional a priori.

Once s_{w,opt} is selected, the coverage α_{w} is given by

α_{w}=tp_{w}(s_{w,opt})

This approach is attractive because it provides
1. a means of coherently incorporating experimental data comparing predicted and actual fitness for some particular fitness estimation method by constructing an empirical ROC curve, e.g., based on Next-Generation Sequencing (NGS) data from harmless virus populations in controlled experiments, and
2. a coherent integration of interpretable cost parameters that capture the trade-offs at play.
   In the instant system, there is a trade-off of the probability of evading screening with the size of the RAT database, and the accompanying global rate of falsely classifying random sequences as hazards. The global false alarm rate, the probability of classifying a random sequence as a hazard, is

$$ℊ = \frac{\vert 𝔻 \vert}{\vert 𝕊 \vert}$$

where [symbol] is the set of all sequences of the same length as the windows, with 20¹⁹ elements for 19-amino-acid protein windows, and 4⁴² elements for 42-base-pair DNA windows. g should be as low as possible due to the accelerating increase in the total amount of DNA synthesized each year.

Any inclusion in the database incurs the same cost in terms of the global false alarm rate of random misclassification, Cₜₚ=C_{fp}:=Cₚ. The cost of a true negative is zero (Cₜₙ=0). The tangency criterion from (4) becomes

$$\frac{dtp}{dfp} = \frac{1 - q}{q} \frac{{C}_{p}}{{C}_{fn} - {C}_{p}}$$

As an aside, due to its relationship to g, Cₚ is inversely proportional to |S|, which is exponential in the length of the window. The window is as long as possible without allowing facile assembly of longer DNA sequences from short sequences that are unscreenable due to being shorter than the window length, which is around 50 base pairs and is an intrinsic physical property of DNA. This constraint is the reason why Cₚ cannot be driven arbitrarily low.

The cost of a false negative C_{fn} has yet to be discussed. C_{fn} is related to the expected exploitability of the false negative by an adversary to increase P(E), which could be the subject of detailed analysis. In particular, it depends on the coverage and the present size of the database. For now, it is treated as an extrinsic parameter to see its effects.

To establish an example, under the simplifying assumption that all k windows have the same ROC, the subscripts w were dropped, and from Eq. 1,

P(E)=(1−α)^{k}

This example shows how the quality of the classifier as captured by its ROC curve affected the optimal choice of parameters, especially k.

The example used | [symbol] _{func}^{w}|=5⁴ for all w, indicating that 5 of the 20 possible amino substitutions were functional at each of 4 positions in each window. It was decided that the maximum Hamming distance, before additional changes cannot conceivably function, was 6. The volume of the Hamming ball for strings of length 19 from an alphabet of 20 with radius 6 is

$$H ( 𝕊 , r ) = {\sum }_{i = 0}^{6} {( 20 - 1 )}^{i} ( \frac{19}{i} ) = 1305752755124$$

q 4.8×10⁻¹⁰ is the ratio (eq. 5). If the value is set as C_{fn}=10⁸Cₚ, that is, neglecting to include a functional variant in the database is 100 million times more costly than including an additional item in the database (conceivable due to the scale of the effect of a successful hazard synthesis),

$$\frac{dtp}{dfp} \approx \frac{1}{q} \cdot \frac{1}{{10}^{8}} \approx 21$$

Though there is no closed form or data for the classifier ROC curve at hand, intuition can be built about the relationship between the “quality” of the classifier and the bound that can be placed on P(E). Qualitatively, a “high quality” classifier makes a clean separation between functional and non-functional variants. It has an ROC curve that is steep near fp=0 and at near fp=1, and reaches high up toward the point (fp, tp)=(0,1). The area under its ROC curve (AUC) is nearer to 1. It might have slopes in the range

$$[ \frac{1}{100} , 100 ] .$$

By contrast, a “low quality” classifier's ROC curve runs closer to the line tp=fp and its AUC is closer to 0.5, meaning that it does not perform much above chance, and might have slopes in the range [2/3, 3/2].

Suppose the high quality classifier reaches the target slope of 21 at fp(sₒₚₜ)=3×10⁻⁶; tp(sₒₚₜ)=0.95. The interpretation is that by covering 0.0003% of the Hamming sphere of reasonable variants around the wild-type sequence, corresponding to a database size of about 1 million, it has accomplished a coverage α=95%, which would be the ideal compromise given the specified balance of costs Cₚ and C_{fn} by definition.

The target bound on the probability of evasion was set at P(E)=0.001, such that an attacker only makes a functional hazard once out of 1000 full orders on average. Using the high quality classifier, the number k of windows that must be covered is

$$k = ⌈ \frac{log ( \text{.001} )}{log ( 1 - \text{.95} )} ⌉ = 3$$

Suppose the low quality classifier is what is available instead. This classifier has no sₒₚₜ such that

$$\frac{dtp}{dfp} = 21.$$

The interpretation is that the low quality classifier cannot be used to reach an optimal compromise between the given balance of costs. Instead, sₒₚₜ was chosen to give a maximum practical database size at 10 million, corresponding to fp(sₒₚₜ)=3.2×10⁻⁵. Because the ROC curve was near the line tp(sₒₚₜ)=fp(sₒₚₜ), the true positive rate tp(sₒₚₜ) cannot be much higher, at say 6×10⁻⁴. The number k of windows that must be covered to accomplish the same bound P(E)=0.001 is

$$k = ⌈ \frac{log ( \text{.001} )}{log ( 1 - 6 \times {10}^{- 4} )} ⌉ = 11510$$

which would not be achievable except for the replicating agents with the largest genomes, and even then, never with this database size.
Key takeaways from the exercise described above were:
The ROC curve of a classifier for the breaking changes fitness approximation could be empirically measured, plotted, and analyzed, for any data set that compares experimentally measured fitness to a given computational tool that predicts protein or DNA functionality, which is readily obtainable.
Explicit costs can be associated with including or not including variants in the database, and these could set the ideal operating point of each classifier.
Use of weak classifiers was possible, but required more windows (greater k) to compensate for their poor true positive rates. k was a function of quality as assessed by the ROC and cost settings only.
Complex interactions existed between cost settings and the optimal operating point. For example, k directly impacted Cₚ via the number of cryptographic operations, which directly translated into DOPRF calls, and the expected exploitability of a single false negative by an adversary to increase P(E) directly impacted C_{fn}. Interactions among these parameters are expected to be convex, and solutions tractable with numerical methods.
There is a minimum average classifier strength necessary to properly bound the probability of evading screening subject to cost constraints.

### 4. Randomly Covering Variants

As the defender approaches perfect knowledge of F(V), it might deterministically choose which windows to protect, because they require the fewest database entries to bound P(E), perhaps arbitrarily close to zero if most functional variants can be collected. Once these fully protectable windows are covered (if one only decides upon the selected windows by this criterion), it would seem that there are no gains to be had by including any other windows. The adversary with oracle knowledge of F(V) knows this and could focus their attention on these regions only, exploiting their superior fitness prediction to find counter-intuitive functional variants that are unlikely to have been screened. Paradoxically, the simpler a hazard is to screen on account of its small number of functional variants, the easier it is for an adversary with superior fitness estimation to evade screening as long as database construction only focuses on deterministically covering certain windows.

A randomized defender strategy can be used increase the expected work an adversary with oracle knowledge of F(V) must do to the point of impossibility by choosing the windows non-deterministically.

### 4.1 Forcing the Adversary to Modify More Windows

This section describes how to make the bounds from Section 2 (above herein) stronger. For this, it was observed that overall there were at most N windows for which there could be entries in [symbol] . On the other hand, due to practical constraints, it may be desirable to add modifications for k of these to [symbol] . In Section 2 an implicit assumption was made that the adversary actually knew which k windows they must modify, but in practice these are actually not known to the adversary.

One approach is to denote the variant collection that the adversary sends as =(νᵢ, ν_{N}) (where it was assumed the windows were not overlapping). The adversary will have modified l values of \vec{ν} when compared to a threat that is of interest to protect against. A first observation is that it is necessary for l≥k: if strictly less than k windows are modified by the adversary, but the original sequence for each such window is in D, the adversary will always be caught.

Next, an adversary that modifies all N windows was considered. Its chance of successfully passing the test with a “fit” sequence is F(\vec{ν}) P(E_{\vec{ν}}) where F(\vec{ν}): F(\vec{ν})=F(ν₁) . . . F(ν_{N}) is the fitness of the actual sequence according to our aforementioned fitness function.

P(E_{\vec{ν}}): P(E_{\vec{ν}}) is the probability that \vec{ν} will not be caught by the RAT.
Setting l=N then P(E_{\vec{ν}}) can be bound exactly as in Section 2. But additionally, the success of an adversary lacking a fitness oracle is influenced by the fitness term, which most likely will be 0 if the whole sequence must be modified.

Towards establishing a bound for k≤l<N, denote by A_{L} the event that the l modifications were chosen by the adversary such that all k windows protected by the adversary were contained. Furthermore, let P(E) be the probability of not being detected in any of the k windows as before and E be the respective event. Because the wild-type sequences were certainly in the database, it must have been that E_{\vec{ν}} ⊆ E ∩ Aₗ as the adversary must at least have passed all k tests and identified the right k out of N windows using l modifications from the wild-type simultaneously. Therefore P(E_{\vec{ν}})≤P(E)·P(Aₗ) and the success probability of the adversary becomes

$${max}_{k \leq l \leq N} F ( {\vec{v}}_{l} ) \cdot P ( E ) \cdot P ( {A}_{l} )$$

where \vec{ν}ₗ is a sequence with l modifications. The standard approach to upper-bound the success probability of the adversary is to find the local maxima with respect to l and then choose k appropriately, which requires the aforementioned function to be differentiable [in particular F(\vec{ν}ₗ)]. Section 2 already gave an explicit bound on P(E) so it becomes necessary to analyze the other terms.

In summary, Example 7 provides a mathematical evaluation of the extreme challenge faced by even a well-equipped adversary when attempting to synthesize a sequence protected by a system of the described invention. The evaluation provided insight into the effectiveness of a screening method of the invention. The larger the fraction of functional variants for a particular window that are present in the database, the lower the odds of evading detection. The more windows that are protected, the lower the odds of evading detection. Given that an adversary able to perfectly predict the function of the resulting sequence—which is a separate and more difficult problem relative to the unsolved problem of perfectly predicting the fitness of a variant for a particular window—will struggle to evade screening, these results suggest that real-world adversaries at risk of including a mutation rendering their sequence nonfunctional have a negligible chance of success as long as sufficient sequences can be included in the database.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms. The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated herein in their entirety herein by reference.

What is claimed is:

1. A method of assessing a biological sequence capable of a preselected function, comprising:
(a) preselecting a biological molecule, wherein the biological molecule is capable of a function of interest;
(b) preparing a testing sequence database comprising a plurality of sequence fragments of the preselected biological molecule, wherein the preselected sequence fragments are a predetermined length;
(c) fragmenting the sequence of one or more test biological molecules into lengths equivalent to the predetermined length of the sequence fragments of the preselected biological molecule in the testing sequence database;
(d) detecting a presence or absence of a sequence match between the sequence of at least one fragment of the fragmented test biological molecules and at least one of the plurality of sequence fragments of the preselected biological sequence, and
(e) acting in response to the detection in (d), wherein the detecting in (d) provides an assessment of the test biological molecule.

2. The method of claim 1, wherein the acting in response to (d) comprises one of more of: preventing synthesis of the test biological molecule, permitting synthesis of the test biological molecule, sequencing one or more polynucleotide molecules, DNA sequencing, DNA molecule design, polypeptide sequence determination, and further sequence identification steps.

3. The method of claim 1, further comprising identifying in the testing sequence database one or more sequence fragments of the preselected biological sequence that match one or more sequence fragments, respectively, of a second biological molecule having a biological function unrelated to the biological function of interest of the preselected biological molecule, and removing the identified sequence fragments(s) from the testing sequence database.

4. The method of claim 1, wherein if the presence of a sequence match is detected in (d) the action comprise preventing synthesis of the test biological molecule.

5. The method of claim 1, wherein a means of preparing the testing sequence database comprises:
(a) screening the plurality of sequence fragments of the preselected biological sequence molecule against at least one control sequence database, wherein the control sequence database comprises a plurality of control sequence fragments of at least one molecule capable of a function of interest unrelated to the function of interest of the preselected biological molecule;
(b) identifying the presence of a match between a sequence fragment in the plurality of sequence fragments of the preselected biological molecule and a sequence fragment in the control sequence database that is a fragment of the biological molecule identified as capable of a function unrelated to the function of interest of the preselected biological molecule; and
(c) removing from the testing sequence database the sequence fragment of the preselected biological sequence identified as matching the sequence fragment of the biological sequence identified as capable of a function of interest unrelated to the function of interest of the molecule capable of the function of interest.

6. The method of claim 1, wherein the preselected biological molecule is a polynucleotide.

7-9. (canceled)

10. The method of claim 1, wherein the preselected biological molecule comprises a polypeptide.

11-13. (canceled)

14. The method of claim 5, wherein the control sequence database comprises a plurality of control sequence fragments of at least one molecule capable of a function of interest unrelated to the function of interest of the preselected biological molecule.

15-17. (canceled)

18. The method of claim 1, wherein the testing sequence database comprises one or more sequence fragments randomly or pseudorandomly selected from sequences of molecules known to be capable of a function different from the preselected molecule's function of interest.

19. (canceled)

20. The method of claim 1, wherein the testing sequence database further comprises sequences that are functional equivalents of the plurality of sequence fragments of the preselected biological molecule.

21. The method of claim 20, wherein a means for identifying the functional equivalents comprises a computational means.

22-25. (canceled)

26. The method of claim 1, wherein the identities of all sequence fragments of one or both of the testing sequence database and the test biological molecule are protected.

27. The method of claim 26, wherein a means of the protecting comprises application of a cryptographic hash function, wherein the cryptographic hash function deterministically maps the sequence data to a bit string of fixed size using a one-way function.

28. The method of claim 27, wherein the application of the cryptographic hash function cannot be reversed without a brute-force search of all possible sequence inputs into the testing sequence database, optionally, wherein the application of the cryptographic hash function further comprises use of one or more information keys that must be accessed to attempt the brute-force search.

29-34. (canceled)

35. A method of identifying a biological sequence capable of a preselected function, comprising:
(a) preselecting a biological molecule, wherein the preselected biological molecule is capable of a function of interest;
(b) preparing a testing sequence database comprising a plurality of sequence fragments of the preselected biological molecule, wherein the preselected sequence fragments are a predetermined length;
(c) fragmenting the sequence of one or more test biological molecules into lengths equivalent to the predetermined length of the sequence fragments the preselected biological molecule in the testing sequence database; and
(d) detecting a presence or absence of a sequence match between the sequence of at least one fragment of the fragmented test biological molecules and at least one of the plurality of sequence fragments of the preselected biological sequence;
wherein a means of preparing the testing sequence database comprises:
(i) screening the plurality of sequence fragments of the preselected biological sequence molecule against at least one control sequence database, wherein the control sequence database comprises a plurality of control sequence fragments of at least one molecule capable of a function of interest unrelated to the function of interest of the preselected biological molecule;
(ii) identifying the presence of a match between a sequence fragment in the plurality of sequence fragments of the preselected biological molecule and a sequence fragment in the control sequence database that is a fragment of the biological molecule identified as capable of a function unrelated to the function of interest of the preselected biological molecule; and
(iii) removing from the testing sequence database the sequence fragment of the preselected biological sequence identified as matching the sequence fragment of the biological sequence identified as capable of a function of interest unrelated to the function of interest of the molecule capable of the function of interest.

36. The method of 35, wherein the preselected biological molecule is a polynucleotide.

37-39. (canceled)

40. The method of claim 35, wherein the preselected biological molecule comprises a polypeptide molecule.

41-50. (canceled)

51. The method of claim 35, wherein the identities of all sequence fragments of one or both of the testing sequence database and the test biological molecule are protected.

52-63. (canceled)

64. A testing sequence database prepared by a method of claim 1.

65. A method of assessing a biological sequence using a testing sequence database of claim 64.

66-70. (canceled)


SEQUENCE LISTING (23 sequences)

SEQ ID NO: 1 - DNA, 105 bp, Artificial Sequence
  Synthetic oligonucleotide
tcatggctgccagagaagtttgagaatgatagtcaggttgtcggtgcaatagtgaatttctggaatactattcatgatacagttcttgcagaaggagggttgaaa

SEQ ID NO: 2 - protein, 35 aa, Artificial Sequence
  Synthetic Polypeptide
SWLPEKFENDSQVVGAIVNFWNTIHDTYLAEGGLK

SEQ ID NO: 3 - protein, 19 aa, Artificial Sequence
  Synthetic Polypeptide
DSQVVGAIVNYWNTIHDTV

SEQ ID NO: 4 - protein, 19 aa, Artificial Sequence
  Synthetic Polypeptide
NSQVVGAIVNFWNTIHDTV

SEQ ID NO: 5 - protein, 19 aa, Artificial Sequence
  Synthetic Polypeptide
DDQVVGAIVDFWNTIHDTV

SEQ ID NO: 6 - protein, 19 aa, Artificial Sequence
  Synthetic Polypeptide
DSQVVGAIVNFWDTIHDTV

SEQ ID NO: 7 - protein, 19 aa, Artificial Sequence
  Synthetic Polypeptide
DSQVVGAIVNFWNTIHNTV

SEQ ID NO: 8 - protein, 19 aa, Artificial Sequence
  Synthetic Polypeptide
NSQVVGAIVNYWNTIHDTV

SEQ ID NO: 9 - protein, 19 aa, Artificial Sequence
  Synthetic Polypeptide
NSQVVGAIVDYWDTIHNTV

SEQ ID NO: 10 - protein, 19 aa, Artificial Sequence
  Synthetic Polypeptide
DSQIVGAIVNFWNTIHDTV

SEQ ID NO: 11 - protein, 19 aa, Artificial Sequence
  Synthetic Polypeptide
DSQVIGAIVNFWNTIHDTV

SEQ ID NO: 12 - protein, 19 aa, Artificial Sequence
  Synthetic Polypeptide
DSQVVGAVVNFWNTIHDTV

SEQ ID NO: 13 - protein, 19 aa, Artificial Sequence
  Synthetic Polypeptide
DSQVVGAIINFWNTIHDTV

SEQ ID NO: 14 - protein, 19 aa, Artificial Sequence
  Synthetic Polypeptide
DSQVVGAIVNFWNTVHDTV

SEQ ID NO: 15 - protein, 19 aa, Artificial Sequence
  Synthetic Polypeptide
DSQVVGAIVNFWNTIHDTI

SEQ ID NO: 16 - protein, 19 aa, Artificial Sequence
  Synthetic Polypeptide
DSEVVGAIVNFWNTIHDTV

SEQ ID NO: 17 - protein, 19 aa, Artificial Sequence
  Synthetic Polypeptide
DSHVVGAIVNFWNTIHDTV

SEQ ID NO: 18 - protein, 19 aa, Artificial Sequence
  Synthetic Polypeptide
DSQVVGAIVNFWNTIQDTV

SEQ ID NO: 19 - protein, 19 aa, Artificial Sequence
  Synthetic Polypeptide
DSQVVGAIVNFWNTIEDTV

SEQ ID NO: 20 - protein, 19 aa, Artificial Sequence
  Synthetic Polypeptide
DTQVVGAIVNFWNTIHDTV

SEQ ID NO: 21 - protein, 19 aa, Artificial Sequence
  Synthetic Polypeptide
DSQVVGAIVNFWNSIHDTV

SEQ ID NO: 22 - protein, 19 aa, Artificial Sequence
  Synthetic Polypeptide
DSQVVGAIVNFWNTIHDSV

SEQ ID NO: 23 - protein, 19 aa, Bacteriophage M13
PQSVECRPFVFGAGKPYEF